(19) United States
(12) Patent Application Publication
(10) Pub. No.: US 2020/0347334 A1
(43) Pub. Date: Nov. 5, 2020

(54) MACROCARRIERS FOR CELL GROWTH IN BIOREACTORS

(71) Applicant: PBS Biotech, Inc., Camarillo, CA (US)

(72) Inventors: Chanyong Brian Lee, Newbury Park, CA (US); Maximilian Lee, Thousand Oaks, CA (US); Sunghoon Jung, Camarillo, CA (US)

(21) Appl. No.: 16/863,819

(22) Filed: Apr. 30, 2020

Related U.S. Application Data
(60) Provisional application No. 62/840,784, filed on Apr. 30, 2019.

(51) Int. Cl. C12M 1/12; C12M 1/00

(52) U.S. Cl. CPC C12M 23/04; C12M 23/20

(57) ABSTRACT

Macrocarriers with flat surface areas which can be easily suspended in liquid using low power input will improve cell attachment and growth in bioreactors. Such macrocarriers can be consistently manufactured to exact size specifications, which can significantly reduce the risk of contamination by small size particulates. The macrocarriers may be flat discs or squares with central bumps on each face to prevent stacking. The macrocarriers may also have outward curved wings from two or four sides to facilitate distribution in moving solution.

### RELATED APPLICATION

The present application claims priority under 35 U.S.C. § 119 to U.S. Provisional Application No. 62/840,784, filed Apr. 30, 2019.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### FIELD OF THE INVENTION

Macrocarriers for bioreactors that can be uniformly manufactured by injection molding to significantly reduce the contamination risk of small plastic particulates. In particular, macrocarriers that provide flat surface area for improved attachment and expansion of adherent therapeutic cells, and can be easily suspended in culture medium in a bioreactor with minimal power input.

### BACKGROUND

With the potential to cure numerous types of serious disease indications, therapeutic cells are poised to revolutionize various therapeutic applications, especially in the fields of regenerative medicine and pharmaceutical cell therapy development. An increasing number of biological drug candidates, such as those derived from mesenchymal or pluripotent stem cells, are currently in development. Many of these cell types are anchorage-dependent, meaning the cells need to attach to a surface before they can grow and expand into available surface space.

Traditionally, anchorage-dependent cells have been cultured at laboratory scale on flat tissue culture plates and flasks whose surface has been pre-treated with special coating to facilitate cell attachment. Once a single anchorage-dependent cell attaches to an open spot on a planar surface, it can easily and quickly grow and expand into the surrounding available space. This 2D, planar approach has been developed and shown to work well for small-scale culturing of anchorage-dependent cells. However, it would be prohibitively expensive to scale up or scale out planar technology to generate the billions or even trillions of therapeutic cells needed for patients at clinical or commercial scale. Instead, 3D platforms such as single-use bioreactors are recognized as a viable solution for large-scale manufacturing of anchorage-dependent cells.

A production bioreactor contains culture medium in a sterile environment that provides various nutrients required to support growth of the biological agents of interest, typically with stirring or other agitation. Stainless steel stirred tanks were originally used for large scale production of biological products in suspension culture. Such conventional bioreactors use mechanically driven impellers to mix the liquid medium during cultivation. The bioreactors can be reused for the next batch of biological agents after cleaning and sterilization of the vessel, which requires a significant amount of time and resources, especially to monitor and to validate each cleaning step prior to reuse for production of biopharmaceutical products. Due to the high costs of construction, maintenance, and operation of conventional bioreactors, single-use bioreactor systems made of disposable plastic material have become an attractive alternative.

Microcarriers are cell attachment substrates commonly used in culturing anchorage-dependent cells in a bioreactor. Most of them are spherical beads, typically large enough to provide sufficient surface area for cell expansion upon attachment, but they are also small enough to allow good suspension and mixing in a bioreactor system. A typical size range for commercially available microcarrier beads is 75 to 250 microns in diameter. These microcarriers consist of either solid beads that permit cell attachment only on the surface or porous beads that permit cell attachment inside the beads, which offer the benefits of more surface area per bead volume for higher cell density processes as well as providing some level of mechanical protection from the shearing effects of the fluid motion if the cells are attached within the pores. However, internal attachment and protection comes at a cost of making it more difficult to detach the cells from microcarriers for harvesting purposes.

Although the most widely used carriers are microcarriers, larger size macrocarriers for use in bioreactors have also been proposed. A general rule of thumb is that a microcarrier has a size less than 300 μm range, while a macrocarrier is larger, though the terms are not always used consistently. For the purpose of the present application, a “macrocarrier” will be deemed to have a size of at least 500 μm (0.5 mm) across its widest point.

In terms of maximizing surface area per gram of material (and thus minimizing weight to improve the ability to suspend in fluid), a spherical microcarrier has “wasted” mass in its center, especially as diameter increases to macrocarrier sizes. While the internally available surface area of porous microcarriers provide more cell attachment points and protection from shear forces compared to spherical microcarriers, they also make dissociation of cells during harvesting more complicated.

Furthermore, cells grown on the curved surface of a spherical microcarrier often appear visually to have much different morphology compared to cells grown on a completely flat surface such as a culture plate. Variations in cell morphology can reduce the total yield of cell expansion or lead to unwanted heterogenous differentiation. Growing anchorage-dependent cells on suspended microcarriers in bioreactors can be the scalable method to achieve large-scale commercial manufacturing of therapeutic cells. However, the spherical shape of currently available microcarriers may affect the quality of cells, compared to the well characterized and consistent growth observed on a flat planar surface.

The current method for manufacturing spherical microcarriers typically involves rapid extrusion of small plastic droplets, which often results in significant variability of particle sizes. In the case of one maker of microcarriers, for instance, the difference in diameter between the 5th and 95th percentiles is 101 microns, with a mean diameter of 190 microns. This large variability in particle size is undesirable from a cell culture standpoint, as it may affect the dynamics of microcarrier suspension, cell attachment efficiency, and most critically, raise the particulates risk of smaller microcarriers ending up in the final drug product.

Part of the cell culture process will include a harvesting step where the therapeutic cells of interest are removed from the surfaces of microcarriers and then separately collected. The smaller the diameter of microcarriers, the harder it will be to separate them from cells, typically through a mesh filtration method. Any microcarriers that are inadvertently collected along with cells are considered to be particulates that can contaminate therapeutic doses, presenting a health risk that could jeopardizes patients' safety and derail a drug product's approval by regulatory agencies. Therefore, it would be beneficial to have a manufacturing process that can reliably produce large quantities of microcarriers of identical size, in order to improve the efficiency of separating microcarriers from cells.

Although there are a number of macrocarrier designs in the art, none as yet has the ability to grow high quality cells in large quantities in bioreactors.

### SUMMARY OF THE INVENTION

The present application discloses several macrocarrier designs for growing cells in bioreactors. Macrocarriers with wide, continuous surface area (primarily flat with some curvature) will improve cell attachment efficiency and mimic the type of growth pattern achieved on the 2D planar platforms commonly used for clinical studies. Macrocarriers that have a wide, continuous surface may be flat or curved disks, having a circular or other peripheral shape. Such shapes will be termed “flat” or “flat surfaced” or “plate-like” for shorthand, though it should be understood that the terms cover more than simply planar surfaces. Compared to sphere-shape microcarriers, plate-like macrocarriers have larger diameters and surface areas per material volume, which are produced using an injection molding process for more consistent and repeatable size control, can greatly minimize the risk of small size contaminants. These macrocarriers preferably possess a disc or square-shaped profile, with either raised wings or asymmetric curvature around the circumference. One macrocarrier design is more in the shape of a hyperbolic paraboloid. All the macrocarriers define flat surface areas available for desired cell attachment and growth.

The embodiments of the present invention address the drawbacks of prior art microcarrier and macrocarrier designs and offer a solution for all of the challenges described above. The proposed macrocarriers feature predominantly flat surface areas to mimic planar 2D tissue culture flasks, allowing for greater surface area per liquid volume displaced, which improves attachment efficiency and cell expansion compared to a similarly sized spherical macrocarrier. At the same time, these macrocarriers are designed to suspend easily in fluid in bioreactors, even without high power input that could cause detrimental shear stress. Furthermore, exemplary macrocarriers featuring predominantly flat surface areas also have three-dimensional aspects that prevent any two flat surfaces from lying completely against one another, or stacking, to ensure that a majority of surface area remains exposed within the bioreactor for cell growth. Most importantly, the size and shape of these macrocarriers can be manufactured consistently as single pieces of identical size by injection mold.

To sum up the advantages of the presently-described macrocarriers, four aspects predominate:

1. The macrocarriers are substantially two-dimensional as opposed to three-dimensional shapes. As mentioned, this increases the surface area per liquid volume displaced for each of the macro carriers, which enhances cell growth efficiency.

2. The macrocarriers preferably have some physical feature which encourages distribution within a bioreactor chamber. The physical feature may be one or more outwardly-directed wings, or peripheral edges which are curved or otherwise shaped to be caught by fluid currents within the bioreactor.

3. To prevent clumping or similar surfaces of macrocarriers stacking together and impeding cell growth, the macrocarriers have shapes which substantially prevent anything but point or line contact between two macrocarriers. These shapes may be in the form of bumps or other raised elements on the broader surfaces of each macrocarrier.

4. Finally, any of the shapes which prevent stacking of the macrocarriers should be limited in size to minimize the interruption of the broader two-dimensional surfaces on which cell growth proliferates.

The following objects of the invention set forth various desirable aspects that may be combined or embodied separately. In general, it is desirable that the macrocarriers:

Demonstrate the ability to suspend in fluid under typical agitation environments (likely varies by vessel volume);

Maximize the surface area per liquid volume displaced;

Maximize the surface area per gram of material;

Minimize the resistance to suspension in liquid flow;

Have a geometry that minimizes the potential for any portion of the macrocarrier to align and stack on top of any portion of another macrocarrier;

Have a geometry that minimizes the potential for the edge of the macrocarrier to cause extensive scraping across the surface of another macrocarrier;

Have a geometry that allows for consistent, repeatable manufacturing of macrocarriers of identical size;

Have a geometry that avoids any portion of the macrocarrier breaking off and becoming small particulates during manufacturing or usage in bioreactors; and

Have the ability to be coated with collagen or other compounds that facilitate cell attachment.

An appreciation of the other aims and objectives of the present invention and an understanding of it may be achieved by referring to the accompanying drawings and the detailed description of a preferred embodiment.

### DESCRIPTION OF THE DRAWINGS

FIGS. 1A-1C are perspective and orthogonal views of a first embodiment of a macrocarrier having a generally planar disc shape;

FIGS. 2A-2C are perspective and orthogonal views of a second embodiment of a macrocarrier having a partial planar disc shape with two oppositely curved “wings” added;

FIGS. 3A-3C are perspective and orthogonal views of a third embodiment of a macrocarrier having a central square plate-like shape with two pairs of oppositely curved “wings” added;

FIGS. 4A-4C are perspective and orthogonal views of a fourth embodiment of a macrocarrier having an undulating plate-like shape with a peripheral edge defining an asymmetric, wave-like curvature;

FIGS. 5A-5D are perspective and orthogonal views of a fifth embodiment of a macrocarrier having a plate-like hyperbolic paraboloid or “saddle” shape and a rounded square peripheral edge in plan view;

FIG. 6 illustrates an exemplary embodiment of a small-scale bioreactor, with vertical-wheel mixing mechanism, in which the macrocarriers described herein can be utilized to optimize scalable cell culture processes.

### DETAILED DESCRIPTION

The present application relates to cell macrocarriers for culturing biological cells, such as pluripotent or multipotent stem cells, wherein the carriers are suspended in a bioreactor. The macrocarriers may be modified by a surface treatment for better cell attachment, controlled growth and easy release. The surface treatment may include applying a coating material such as collagen, gas plasma treatment, corona discharge treatment or combinations thereof.

Certain microcarrier designs utilize honeycomb or lattice-like 3D structures with an array of planar surfaces, much like shelves. For instance, U.S. Patent Publication No. 2012/0156777 to Rangarajan discloses a number of macrocarriers with indents or cups formed therein. Rangarajan also suggests using planar discs die-cut from suitable film. There are disadvantages to both purely planar macrocarriers which can stack directly on top of each other, as well as forming them by cutting, which may contaminate the culture medium with particulates from the cut edges. U.S. Pat. No. 4,842,920 to Banai and U.S. Patent Publication No. 2016/0046898 to Lee also disclose 3D macrocarriers with multiple internal planar surfaces on which cells may grow. Separating the cells grown on these macrocarriers is difficult given the intricate geometry.

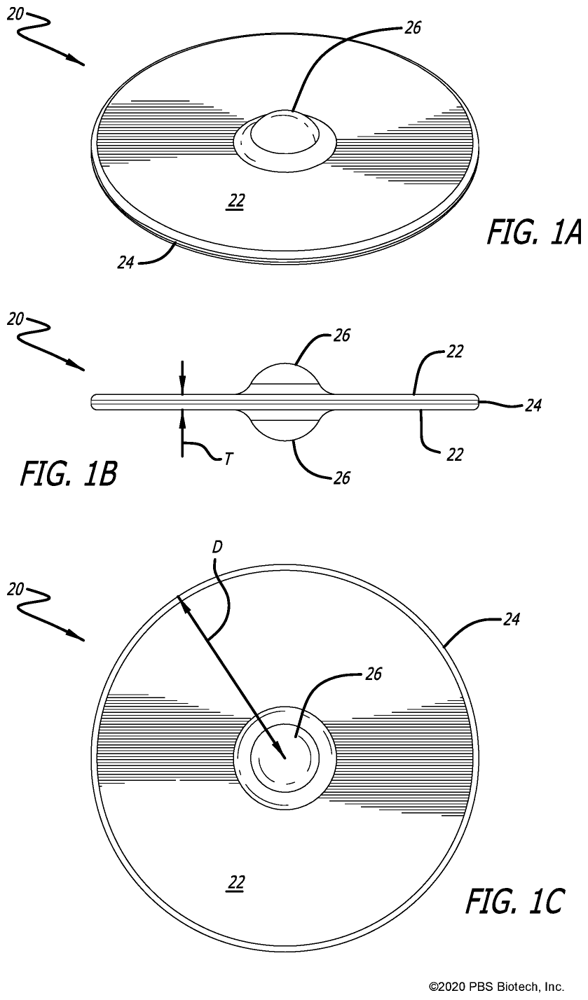

The macrocarriers disclosed herein are generally flat and disc or square-shaped or plate-like, but with varying number of curved wings or asymmetric curvature, as exemplified by the embodiments described below (FIGS. 1-5). Being plate-like maximizes flat surface area available for adherent cells to attach and grow with uniform morphology while minimizing total mass and weight of the macrocarriers. Minimizing weight will make it easier for the macrocarriers to be completely and constantly suspended in liquid medium with minimal power input to a bioreactor's mixing mechanism. A preferred macrocarrier design maximizes surface area available for cells to attach and grow, per liquid volume displaced as well as per gram of material. For instance, the diameter would be in the range of 0.5 to 5 millimeters and the thickness at least 0.02 millimeters. The diameter, thickness, and material of construction can all be adjusted to optimize the ability of the macrocarriers to suspend in fluid in bioreactors based on different cell culture process parameters.

Plate-like means that there are two generally parallel opposed faces separated by a peripheral edge. Plate-like is also defined as a body with a peripheral shape and a thickness, where a ratio of the minimum dimension across the body to the thickness is 0.4-10% with a preferred ratio within 1-4%. If the diameter or distance across the wide axis of the macro carrier is between 0.2-5 mm, with a thickness of at least 0.02 mm, the minimum ratio possible is thus 0.004 (0.02/5), while the maximum ratio is 0.1 (0.02/0.2). It also should be noted that “generally parallel” is inclusive of some variance, such as a wedge-shaped body. Preferably, the thickness around the peripheral edge may vary by up to 20%. For example, given a body with a diameter of 5 mm, the thickness could vary from 0.2 mm 0.04 mm.

Compared to microcarriers, the larger diameters and overall dimensions of macrocarriers can be beneficial for consistently manufacturing them to be identical sizes, most likely through injection molding. By injection molding a macrocarrier as a single piece that does not require assembly, the risk of any portion of the macrocarrier breaking off and forming undesirable particulates is greatly reduced (this of course also depends on material of construction and the thickness of the macrocarrier).

These newly proposed macrocarrier designs, to be suspended in bioreactors, combine the benefits of 2D and 3D cell culture: a flat surface for cells to easily attach to and spread out as they grow, and an overall shape that can be suspended easily in fluid inside scalable bioreactors, respectively.

With reference now to FIGS. 1A-1C, a first embodiment of a macrocarrier 20 is a simple flat, planar disc having opposite faces 22 circumscribed by a circular periphery 24 about a central vertical axis. Both faces 22 feature raised circular bumps 26 in the center thereof. In one embodiment, the bumps 26 are small, shallow and hemispherical. By “small, shallow” is meant that the diameter of the bumps 26 is small compared to the minimum dimension across the disc and has a height that is shallow relative to the thickness T of the disc. For instance, the bumps 26 have a diameter no greater than 25% of the diameter D of the disc and a height rejected from each side of no greater than twice the thickness T of the disc.

Compared to a spherical microcarrier, there is much more flat or extended surface area available for cell attachment and expansion, and less wasted central mass. As an example, if the diameter D of the disc is set at 0.5 mm and the thickness T at 0.02 mm, then the total flat surface area available for adherent cells to attach and grow would be 0.4213 mm². In a preferred embodiment, the diameter D of the disc is between about 0.2-5.0 mm, and the thickness T ranges between 0.016-0.024 mm.

The raised bumps 26 not only prevent any two macrocarriers 20 from stacking directly on top of each other during static conditions, but also prevent the macrocarriers from lying completely flat on a surface, such as a single-use bioreactor's vessel bottom. The macrocarriers will be slightly tilted, with the bump on one side and a portion of the disc's edge touching a surface simultaneously. Therefore, flowing fluid will hit the raised edge of the disc and invariably cause the macrocarriers to flip and tumble and become suspended in liquid. However, with this design, the flat portions around the bumps of two different macrocarriers can still stack on top of each other, or the edge of one macrocarrier can scrape along the surface of another macrocarrier, with both scenarios causing potential cell damage or unwanted cell aggregation.

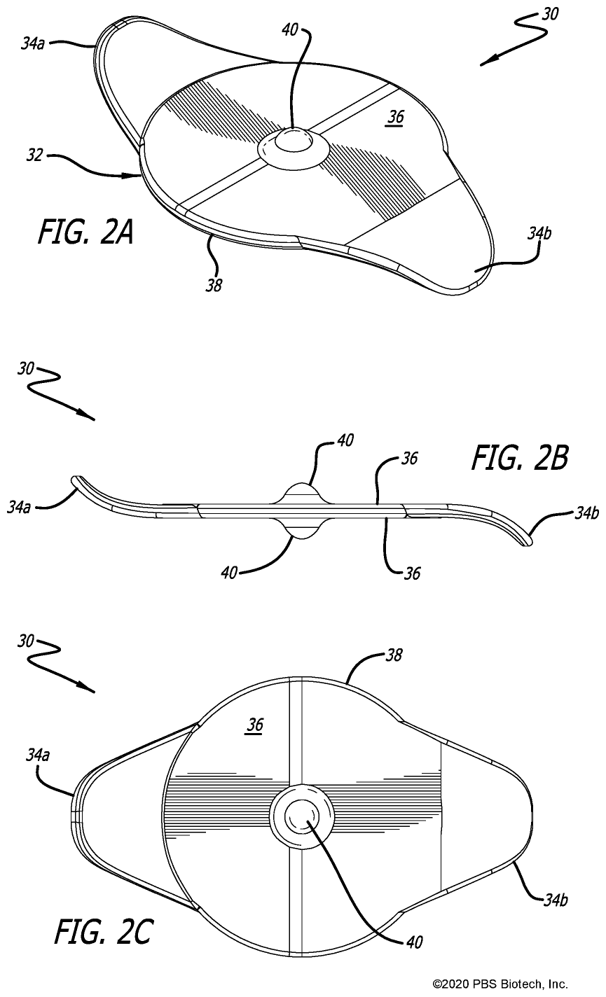

FIGS. 2A-2C illustrate a second embodiment of a macrocarrier 30 that retains the flat, disc-shaped body defined by a pair of opposed planar faces 36 and a partial circular peripheral edge 38. Once again, the macro carrier 30 has a pair of central bumps 40 projecting from both faces 36, and also introduces two raised “wings” 34a, 34b that protrude from the opposite edges of the central disc 32. These two wings 34a, 34b curve in opposite axial directions on either side of the disc and their primary function is to provide raised surface areas for contacting flowing fluid. Compared to the previous flat disc shape 20 of FIGS. 1A-1C, the raised wings 34a, 34b will cause the macrocarriers to be more easily swept along and tumble in fluid, leading to better suspension in liquid and preventing the macrocarriers from settling at the bottom of the bioreactor vessel. However, as the wings 34a, 34b are added to the edges of the disc 32 and curve in opposite directions, a majority of flat surface area is still available for potential stacking or scraping between two macrocarriers. Also, with a width dimension of 0.5 mm (measured as the widest distance between the tips of both wings) and thickness of 0.02 mm, the total surface area is 0.212 mm², which is a 49.7% decrease from the previous flat disc design with the same diameter and thickness. A critical manufacturing factor to consider is the potential risk of a wing breaking off and becoming a particulate in fluid.

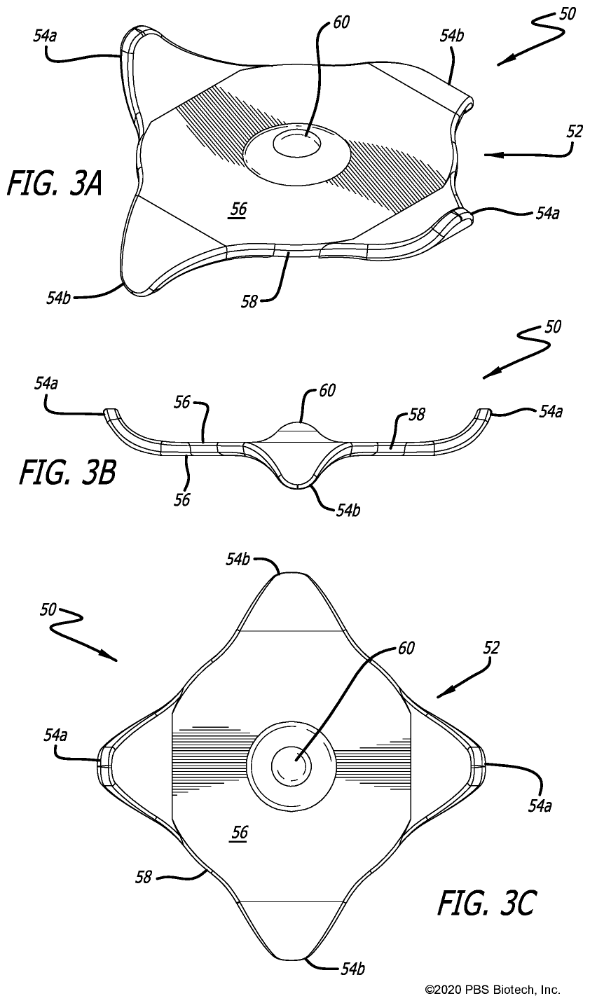

FIGS. 3A-3C illustrates a further variation of the macrocarrier design described in FIGS. 2A-2C. The primary shape of the macro carrier 50 is a flat disc or plate-like circular or square body 52 having two pairs of outer curved wings 54a, 54b. The total of four wings 54a, 54b raises the probability of fluid flow contact and thus tumbling and suspension. Each wing curves in the opposite direction compared to a 90° adjacent wing. That is, there is a first pair of diametrically-opposed wings 54a curled in one axial direction between a second pair of diametrically-post wings 54b curled in the opposite axial direction. Compared to the previous two designs, the orientation of the four wings makes it more difficult for two macrocarriers to align along the edges of the disc and stack, although the protruding wings 54a, 54b could still cause scraping. However, if the width dimension from the tips of any two opposing wings is measured to be 0.5 mm and the thickness is 0.02 mm, then the overall surface area for cell attachment and growth is 0.2846 mm², which is a decrease of 32.45% compared to the basic disc shape of FIGS. 1A-1C.

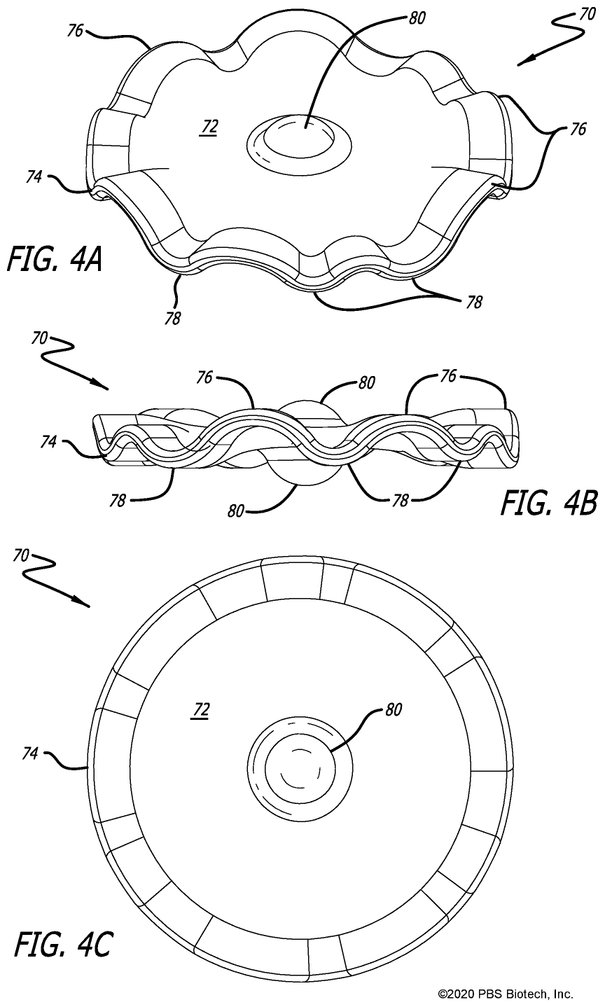

FIGS. 4A-4C illustrate a more drastic variation of the basic flat disc shape of FIGS. 1A-1C. In this design, the entire circumference of the flat disc is curved or grooved in an asymmetric, wave-like manner, with the peaks and valleys of the curves in the direction of the disc's vertical axis. More specifically, the macro carrier 70 has opposite faces 72 and an undulating peripheral edge 74. The peripheral edge 74 includes alternating upwardly-curved portions 76 and downwardly-curved portions 78. Once again, central bumps 80 project from the opposite faces 72.

The concave portion of each curved portion 76, 78 around the peripheral edge 74 will “catch” flowing fluid and promote tumbling and suspension of the macrocarriers in liquid, as well as prevent settling. By making the height of each curved portion 76, 70 and the spacing between them asymmetric to each other, the probability of two macrocarriers stacking on top of each other, even just along their edges, is minimized. That is, some of the curved portion 76, 78 are larger than others and the pattern is non-repeating around the peripheral edge 74 to minimize the chances of two macrocarriers 70 nesting against one another. Furthermore, the overall surface area of this design, with diameter of 0.5 mm and thickness of 0.02 mm, is 0.4644 mm², which is an increase of 10.23% compared to the basic flat disc design.

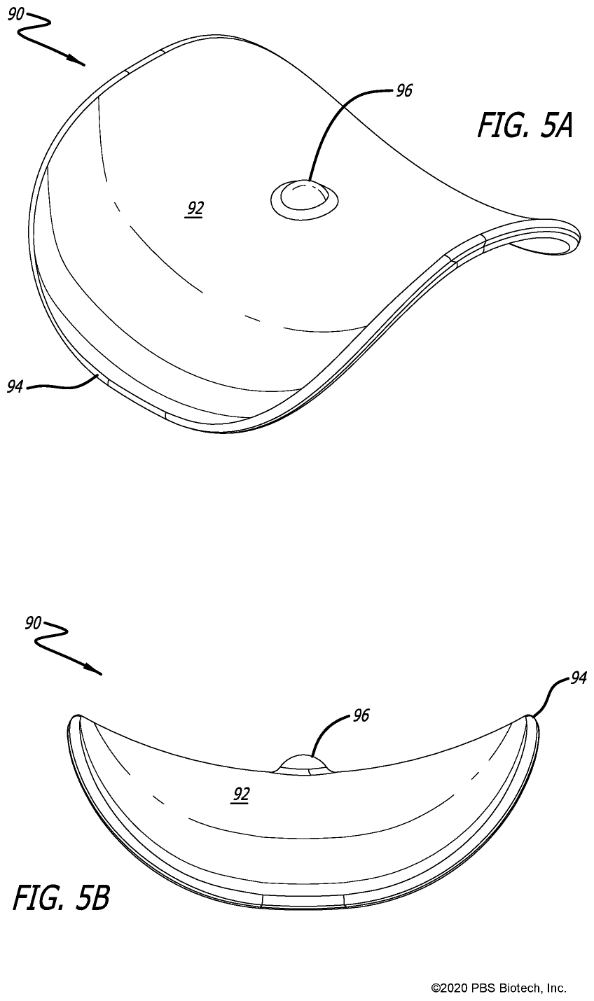

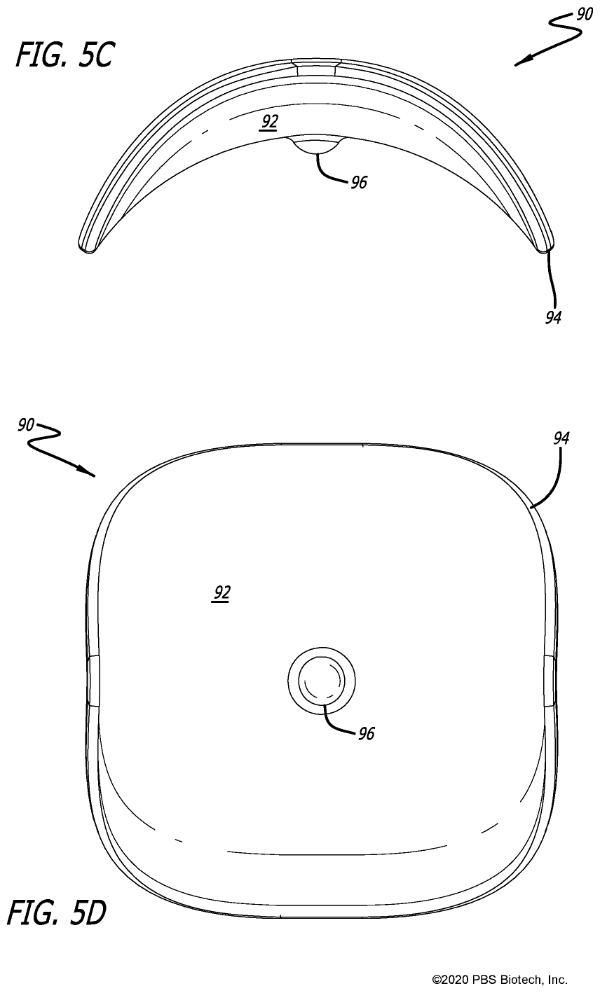

FIGS. 5A-5D illustrate a different type of macrocarrier 90 compared to the flat disc that is the basis for the previous four designs. In this variation, the macrocarrier 90 is in a constantly curved hyperbolic paraboloid or “saddle” shape with no planar areas. Specifically, the macrocarrier 90 has a pair of opposed faces 92 surrounded by a peripheral edge 94, and also a pair of oppositely-directed bumps 96 projecting axially from each face 92. As seen from above in FIG. 5D, the plan view shape of the macrocarrier 90 is a rounded square, although it may likewise be formed to be circular or oval. The peripheral edge 94 undulates up and down, with diametrically-opposed sides curving in the same direction. Each pair of diametrically opposed portions of the peripheral edge 94 that curve in the same direction form wings that help distribute the macrocarrier 90 within the currents formed in the bioreactor. That is, the wings formed by the peripheral edge 94 act in a similar manner as the protruding wings 54a, 54b seen in the embodiment of FIGS. 3A-3C.

Compared to the basic flat disc in FIGS. 1A-1C, this saddle-shaped macro carrier 90 with the same 0.5 mm diameter and 0.02 mm thickness results in a total surface area of 0.5818 mm², which is a substantial increase of 38.10% compared to the flat disc. The central bumps 96 on both sides will prevent direct stacking on top of each other, while the curved edges prevent settling and promote tumbling by fluid flow contact. Indeed, in this configuration there is only one position in which two macrocarriers 90 can potentially stack, much like Pringles chips in a can, which is prevented by the bumps 96 on both sides. For this design, the thickness of the macrocarrier may have to be optimized to prevent any potential breakage along the central axes.

It should be understood that various aspects of the macrocarriers described herein, such as the shape, orientation, and placement of wings or curves, can be interchanged and thus other permutations not illustrated are contemplated. When considering an “ideal” macrocarrier design, there may be certain trade-offs depending on which benefits are most desirable for a particular cell culture process. For example, adding more curved wings improves the ability of the macrocarriers to become suspended in liquid but, depending on construction, may also increase the possibility of wings breaking off and forming undesirable particulates. Making the macrocarriers larger to provide more surface area will increase weight, requiring more power input from the bioreactor mixing mechanism to suspend, and may also restrict movement through tubing during various cell culture processes. Increasing the height or narrowness of various curvatures may provide better contact surfaces to promote suspension, but may result in more “dead zones” or pockets of liquid between settled macrocarriers during a medium exchange process, where the goal is to remove as much liquid as possible. Ultimately, the design must allow for repeatable manufacturing of identically-sized macrocarriers in order to minimize the risk of unwanted particulates.

The macrocarriers may be formed from a variety of materials, preferably moldable polymers such as polystyrene (PS), polyethylene (PE), polycarbonate (PC), and polypropylene (PP). The flat surfaces may be treated so as to enhance cell growth. For example, the macrocarriers may be immersed in a collagenous solution prior to use so as to coat the flat surfaces with collagen or other materials. Some cell growth processes, however, require the absence of any animal cell components, in which case the flat surfaces may be roughened somewhat using gas plasma treatment, corona discharge treatment or combinations thereof. It should be understood that each of the various macrocarriers described herein can be treated in the same manner.

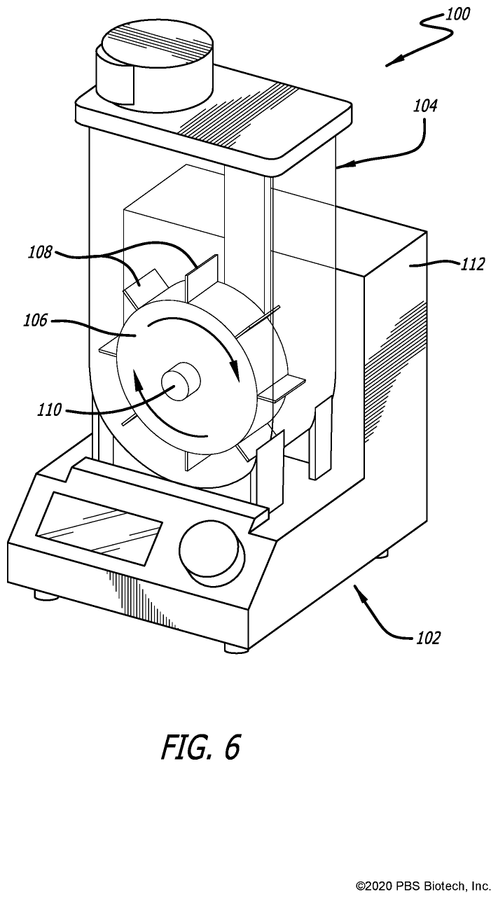

Finally, FIG. 6 illustrates an exemplary embodiment of a small-volume bioreactor 100 in which the macrocarriers described herein can be utilized. The bioreactor 100 comprises a base unit 102 supporting a disposable container 104. The container 104 preferably has a generally rectangular upper section and a semi-cylindrical lower section, as shown. A mixing or agitating wheel 106 is mounted wholly within the container 104 for rotation within the semi-cylindrical lower section. Preferably, the wheel 106 features a series of vanes 108 on its exterior force during the solution within the container 104, and also preferably includes inner vanes (not shown). The wheel 106 rotates about a horizontal axis on hubs 110 secured to the front and/or back walls of the container 104 (i.e., only one wheel hub 110 may be secured to the container 104). In a preferred embodiment, the base unit 102 includes an upstanding cabinet 112 within which is housed a drive system including rotating magnets (not shown). Corresponding magnets or ferromagnetic material mounted around the wheel 106 allow coupling of the drive system to enable rotation of the wheel from outside the container 104, thus eliminating seals and the like which might contaminate the solution within the container. In a preferred embodiment, the volume capacity of the container 104 is between 0.05-1.0 L, although the system can be scaled up for larger capacities.

The illustrated bioreactor 100 is for use inside CO₂ incubators, which are typically run with temperature control and with a fixed percentage of CO₂ in air. Consequently, independent pH and DO controls for the bioreactor 100 are not necessary.

Contemplated options for the macrocarriers include:

The possibility for uneven distribution of mass in order to encourage certain orientation or behavior;

An optimal number of wings or curvatures around the circumference of the macrocarriers to promote fluid flow contact and therefore tumbling in liquid;

The possibility for uneven distribution and/or asymmetric shaping of wings or curvatures; and

An optimal number/thickness for different diameters of macrocarriers.

It is understood that the foregoing examples are considered illustrative only of the principles of the invention. Further, since numerous modifications and changes will readily occur to those skilled in the art, it is not desired to limit the invention to the exact construction and operation shown. Accordingly, all suitable modifications and equivalents may be resorted to, falling within the scope of the invention.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

It is claimed:

1. A macrocarrier for a cell culture growth process, comprising:
a molded plate-like body having opposed faces separated by a peripheral edge, the body having a thickness T that is between about 0.4-10% of the minimum dimension from one side of the peripheral edge to the other, the body defining a vertical axis perpendicular to the thickness and further including a vertical bump projecting from each face to prevent two macrocarriers from contacting one another across entire adjacent faces.

2. The macrocarrier of claim 1, wherein the body is a disc or square having a minimum dimension from one side of the peripheral edge to the other of greater than 0.2 mm and a thickness of greater than 0.02 mm.

3. The macrocarrier of claim 1, wherein the bumps are hemispherical.

4. The macrocarrier of claim 1, wherein two wings protrude from diametrically opposite sides of the peripheral edge of the disc and curve in the same direction from the body.

5. The macrocarrier of claim 1, wherein four wings protrude from the peripheral edge of the disc, angularly spaced at 90° to each other, and each wing curves in the same direction as the wing diametrically opposite thereto.

6. The macrocarrier of claim 1, wherein the entire peripheral edge of the disc has asymmetric, wave-like curves undulating in the direction of the vertical axis of the disc.

7. The macrocarrier of claim 1, wherein the body has a constantly curved hyperbolic paraboloid or saddle shape without any planar areas.

8. The macrocarrier of claim 1, further including a surface treatment to enhance cell growth.

9. The macrocarrier of claim 8, wherein the surface treatment is a coating of collagen.

10. The macrocarrier of claim 8, wherein the surface treatment is chosen from the group consisting of:
gas plasma treatment, and
corona discharge treatment.

11. A macrocarrier for a cell culture growth process, comprising:
a molded plate-like body having opposed faces separated by a peripheral edge and a thickness T across the opposed faces, the body defining a vertical axis perpendicular to the thickness and further including a vertical bump projecting from each face to prevent two macrocarriers from contacting one another across entire adjacent faces, the body further including at least two wings formed at diametrically opposite sides of the peripheral edge that curve in the same direction from the body.

12. The macrocarrier of claim 11, wherein the body has four wings formed around the peripheral edge of the body, angularly spaced at 90° to each other, and each wing curves in the same direction as the wing diametrically opposite thereto.

13. The macrocarrier of claim 12, wherein the body has a constantly curved hyperbolic paraboloid or saddle shape without any planar areas, and the wings are formed by the peripheral edge.

14. The macrocarrier of claim 13, wherein the body has a substantially rounded square shape in top plan view.

15. The macrocarrier of claim 11, wherein the body has a substantially rounded square shape in top plan view.

16. The macrocarrier of claim 11, wherein the body has a thickness T that is between about 0.4-10% of the minimum dimension from one side of the peripheral edge to the other.

17. The macrocarrier of claim 16, wherein the body is a disc or square having a minimum dimension from one side of the peripheral edge to the other of greater than 0.2 mm and a thickness of greater than 0.02 mm.

18. The macrocarrier of claim 11, wherein the bumps are hemispherical.

19. The macrocarrier of claim 11, wherein the body has a constantly curved shape without any planar areas.

20. The macrocarrier of claim 11, wherein the body is planar disc or square with the wings projecting outward from the peripheral edge.

21. The macrocarrier of claim 11, further including a surface treatment comprising a coating of collagen to enhance cell growth.

22. The macrocarrier of claim 11, further including a surface treatment to enhance cell growth chosen from the group consisting of:
gas plasma treatment, and
corona discharge treatment.
